(19) United States
(12) Patent Application Publication
(10) Pub. No.: US 2022/0098574 A1
(43) Pub. Date: Mar. 31, 2022

(54) METHODS OF SEPARATING LONG POLYNUCLEOTIDES FROM A COMPOSITION

(71) Applicant: Flagship Pioneering Innovations V, Inc., Cambridge, MA (US)

(72) Inventors: Jennifer Louise Schmitke, Belmont, MA (US); Robert John Lyng, Lexington, MA (US); Vishwesh Ashok Patil, Arlington, MA (US); Can Sarisozen, Foxborough, MA (US); Marcus Ian Gibson, Belmont, MA (US)

(73) Assignee: Flagship Pioneering Innovations V, Inc., Cambridge, MA (US)

(21) Appl. No.: 17/425,545

(22) PCT Filed: Jan. 29, 2020
(86) PCT No.: PCT/US2020/015577

§ 371 Date: Jul. 23, 2021

Related U.S. Application Data
(60) Provisional application No. 62/798,038, filed on Jan. 29, 2019.

(51) Int. Cl. C12N 15/10; B01D 61/14; B01D 61/18; B01D 61/22

(52) U.S. Cl. CPC C12N 15/1017; B01D 61/145; B01D 61/18; B01D 2313/24; B01D 2311/14; B01D 2313/20; B01D 61/22

(57) ABSTRACT

One aspect of the invention provides a method of separating long polynucleotides from a composition. The method includes: introducing a composition including one or more long polynucleotides into a container including at least one boundary defined by a filter comprising a plurality of pores, wherein the pores have a smaller cross-sectional dimension than specified for the long polynucleotide's molecular weight; and applying elevated hydraulic pressure to the composition, thereby causing at least some of the one or more long polynucleotides to pass through the pores.

### CROSS-REFERENCE TO RELATED APPLICATION

This application claims the benefit of priority to U.S. Provisional Patent Application Ser. No. 62/798,038, filed Jan. 29, 2019. The entire content of this application is hereby incorporated by reference herein.

### BACKGROUND OF THE INVENTION

As the fields of biologics and gene editing develop into the clinical therapies, purity of the therapeutic grows in importance.

### SUMMARY OF THE INVENTION

The present invention includes methods of separating long polynucleotides from a composition.

One aspect of the invention provides a method of separating long polynucleotides from a composition. The method includes: introducing a composition including one or more long polynucleotides into a container including at least one boundary defined by a filter comprising a plurality of pores, wherein the pores have a smaller cross-sectional dimension than specified for the long polynucleotide's molecular weight; and applying elevated hydraulic pressure to the composition, thereby causing at least some of the one or more long polynucleotides to pass through the pores.

Another aspect of the invention provides a method of separating a long polynucleotide from a composition. The method includes: (a) providing a composition comprising a long polynucleotide, (b) disposing the composition within a container including at least one boundary comprising a plurality of pores having a smaller cross-sectional dimension than specified for a molecular weight of the long polynucleotide; and (c) subjecting the composition to elevated hydraulic pressure within the container under conditions to cause the long polynucleotide to pass through the pores, thereby separating the long polynucleotide from the composition.

These aspects can have a variety of embodiments. The long polynucleotides can have a length of about 1 kilobase or greater. The long polynucleotides can have a length selected from the group consisting of: ≥950 kilobase, ≥900 kilobase, ≥850 kilobase, ≥800 kilobase, ≥750 kilobase, ≥700 kilobase, ≥650 kilobase, and ≥600 kilobase.

The composition further can include a second component. The second component may not pass through the pore.

The filter can be positioned within the container between an inlet and an outlet and the composition can be flowed over the filter. The composition can be flowed laterally over the filter.

The elevated hydraulic pressure can be between about 0.1 and about 30 pounds per square inch (psi), inclusive. The elevated hydraulic pressure can be below a pressure that would impact the stability of other components of the compositio.

The long polynucleotides can be sheared during passage through the pores.

The pores can have an effective molecular weight cutoff of about 1,000 kDa.

The pores can have a maximum cross-sectional dimension between about 50 nm and about 150 nm.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 depicts tangential flow filtration according to an embodiment of the invention.

FIG. 2 depicts of generalized proportions of long polynucleotides (e.g., messenger RNA) and lipid-based nanoparticles (LNPs).

FIG. 3 depicts the passing of long polynucleotides through the pores of a filter while long polynucleotides encapsulated within LNPs are retained according to embodiments of the invention.

FIG. 4 depicts mRNA concentrations in the retentate vs. wash cycles.

FIG. 5 depicts encapsulation efficiency (EE) and recovery for different lipid-based nanoparticles (LNPs) encapsulating different cargos. LNP_3, LNP_5 and LNP_11 were loaded with sgRNA (100 nt, ˜34 kDa). The LNP+mRNA formulation contained mRNA with 1,500 kDa size and 4500 kb.

FIG. 6 depicts EE and recovery values of an optimized LNP formulation encapsulating mRNA, processed by an embodiment of the invention.

### DETAILED DESCRIPTION

In one aspect, the present invention is directed to, among other things, methods of purifying polynucleotides or other components from a mixture by tangential flow filtration.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The term “assembled LNP” or “assembled lipid nanoparticle” are used interchangeable and as used herein refer to a lipid nanoparticle comprising at least one mRNA.

The term “boundary” as used herein refers to an edge that defines an extent past which a substance (e.g., long polynucleotides within a composition) cannot pass, at least without a reduction in concentration or physical deformation. A boundary may exist within a container such that the boundary limits passage from a first chamber to a second chamber. A boundary may also exist on an external surface such that boundary limits flow of a substance into or out of the container. The boundary may be in-line with a direction of flow of the composition or may be lateral to a direction of flow.

The term “container” as used herein refers to any receptacle that holds a substance such as a fluid or liquid. Exemplary containers can be finite such as a bucket, a syringe, and the like. Exemplary containers can also be continuous such as cylinders, tubing, and the like that can have arbitrary lengths.

The term “cross-sectional dimension” as used herein refers to the largest dimension transverse to a longest axis. For example, the cross-sectional dimension of a rectangular cuboid having a length of 10, a width of 4, and a height of 3 would have a cross-sectional dimension of 5 (the diagonal of the width-by-height).

The term “hydraulic pressure” as used herein is defined as force applied by a liquid over a unit area.

The term “long polynucleotide” as used herein is defined as a chain of nucleotides having a length of 1000 nucleotides or more. Furthermore, nucleic acids are polymers of nucleotides. Thus, nucleic acids and polynucleotides as used herein are interchangeable.

The term “pore” as used herein refers to an opening in a filter through with a filtrate can pass through, but which retains (at least in part) a retentate.

### Mixtures Containing Polynucleotides

The present invention includes methods of purifying free polynucleotides from other components, where either the polynucleotides or the other components are suitable for administration as a pharmaceutical product based on tangential flow filtration (TFF). In some embodiments, the mixture comprises polynucleotides and proteins. In some embodiments, the mixture comprises non-protein components, such as nanoparticles, and polynucleotides. In some embodiments, the mixture comprises any combination of components and polynucleotide.

Prior to the present invention, two main buffer exchange/purification/concentration methods are used in the technique of processing polynucleotide mixtures comprising formulations. The first involves the dialysis of the polynucleotide mixtures against PBS for buffer exchange and removal of ethanol from the dispersion. However, when long polynucleotides are present (˜1500 kDa), this method cannot be used because the largest molecular weight cutoff for dialysis membranes is 300 kDa, far smaller than the polynucleotides. While high pressures might push long polynucleotides through the 300 kDa membrane, the pressure levels required for effective purification will negatively impact nanoparticle stability and cause nanoparticle breakdown during the membrane pass. Furthermore, the removal process takes significantly longer time, generally requiring 24-48 h of dialysis.

The second method involves using centrifugal ultrafiltration devices. In this method, the nanoparticles are pushed through a membrane by applying extreme g-force. But if the g-force is too low, the removal of free polynucleotides is limited. If the force is high, then stability of the nanoparticles is negatively affected, e.g., the nanoparticles are crushed.

The present invention is, in part, based on the discovery that tangential flow filtration is surprisingly effective to remove components, e.g., byproducts, in particular, guide RNAs or other free polynucleotides, from polynucleotide mixtures. As described herein, tangential flow filtration can effectively remove components, e.g., byproducts such as gRNA or other free polynucleotides, while still maintaining the integrity of polynucleotides or the other component. Thus, the present invention includes a more effective, reliable, and safer method of purifying RNA, assembled nanoparticles, or other components from large scale manufacturing and processing of therapeutics.

### LNPs

Lipid-based nanoparticles (LNPs) are one of the most effective non-viral transfection strategies for m vivo delivery of nucleic acid-based therapeutics, including RNA-based therapeutics. LNPs are typically composed of four main lipid types, a cationic or ionizable lipid, a neutral helper lipid, cholesterol for structural integrity, and sterically stabilizing lipid. Cationic an ionizable lipids contain a functional group (e.g., an amine group) that carries a permanent positive charge or that can be positively charged at low pH values and, thus, complex the negatively charged RNA (e.g., mRNA) and different guide RNAs (dual or single). Sterically stabilizing lipids are usually PEG-lipid conjugates (e.g., PEG-DMG), which cover the surface of the LNPs and shield overall surface charges (positive or negative), making the surface hydrophilic. In vivo applications of sterically stabilizing lipids prevent opsonization and increase the longevity of the LNPs in the bloo.

In some embodiments, the present invention includes a process of encapsulating messenger RNA (mRNA) in lipid nanoparticles by methods known to those of skill in the art, such as by mixing a mRNA solution and a lipid solution. For example, see US20180263918.

Effective encapsulation of the nucleic acids is important because free (or naked) RNA molecules would have a significantly shorter half-life in the blood circulation (usually measurable in minutes). Moreover, non-encapsulated mRNA molecules that are not effectively removed from drug products may be immunogenic. Thus, removal of free/non-encapsulated nucleic acids, e.g., free guide RNAs, mRNAs, etc., and purification of LNPs are equally important as encapsulating the mRNAs (e.g., those having greater than about 1,400 bases, about 1,350 bases, about 1,300 bases, about 1,250 bases, about 1,200 bases, about 1.150 bases, about 1,100 bases, about 1,050 bases, about 1,000 bases, about 950 bases, about 900 bases, about 850 bases, about 800 bases, about 750 bases, about 700 bases, about 650 bases, about 600 bases, and the like).

In some embodiments, the present invention includes a composition comprising LNPs associated with at least one mRNA (e.g., greater than about 1,400 bps, about 1,350 bps, about 1,300 bps, about 1.250 bps, about 1,200 bps, about 1,150 bps, about 1,100 bps, about 1,050 bps, about 1,000 bps, about 950 bps, about 900 bps, about 850 bps, about 800 bps, about 750 bps, about 700 bps, about 650 bps, about 600 bps, and the like) and one guide RNA (e.g., less than 100 bps) (e.g., LNP+mRNA+gRNA is an assembled LNP), wherein greater than about 90% of the LNPs have an individual particle size of less than about 100 nm (e.g., less than about 95 nm, about 90 nm, about 85 nm, about 80 nm, about 75 nm, about 70 nm, about 65 nm, about 60 nm, about 55 nm, or about 50 nm) and greater than about 70% of the LNPs encapsulate at least one mRNA and/or at least one guide RNA within each individual particle. In some embodiments, greater than about 95%, 96%, 97%, 98%, or 99% of the LNPs have an individual particle size of less than about 100 nm (e.g., less than about 95 nm, about 90 nm, about 85 nm, about 80 nm, about 75 nm, about 70 nm, about 65 nm, about 60 nm, about 55 nm, or about 50 nm). In some embodiments, substantially all of the LNPs have an individual particle size of less than about 100 nm (e.g., less than about 95 nm, about 90 nm, about 85 nm, about 80 nm, about 75 nm, about 70 nm, about 65 nm, about 60 nm, about 55 nm, or about 50 nm). In some embodiments, greater than about 75%, 80%, 85%, 90%, 95%, 96%, 97%, 98%, or 99% of the assembled LNPs encapsulate at least one mRNA within each individual particle. In some embodiments, substantially all of the LNPs encapsulate at least one mRNA and/or at least one guide RNA within each individual particle. In some embodiments, a composition according to the present invention includes at least about 1 mg, 5 mg, 10 mg, 100 mg, 500 mg, or 1000 mg of encapsulated mRNA and/or guide RNA. In some embodiments, some of the LNPs will not include a payload, will not include gRNA, will not include mRNA, will only include gRNA, will only include mRNA, and the like

### Physical Characteristics of Polynucleotides

Embodiments of the invention exploit the physical characteristics of polynucleotides such as RNA relative to other components, e.g., lipid-based nanoparticles (LNPs).

Without being bound by theory, Applicant believes that long polynucleotides are relatively narrow and have high aspect ratios. For example, polynucleotides are believed to have a maximum cross-sectional width of about 3 nm regardless of length, which can be on the order of about 300 nm for about a 1,000 base pair polynucleotide. These proportions of long polynucleotides 202 are depicted conceptually in FIG. 2.

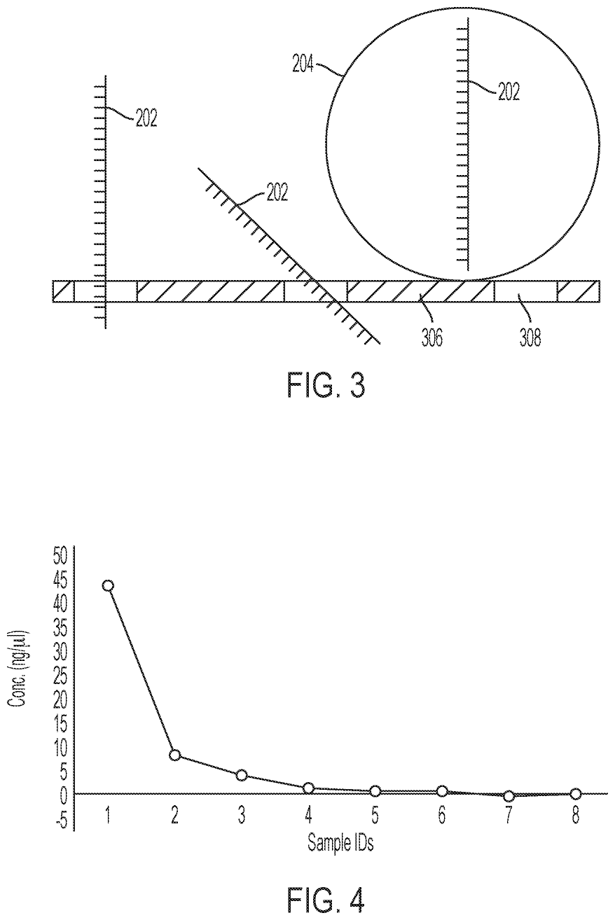

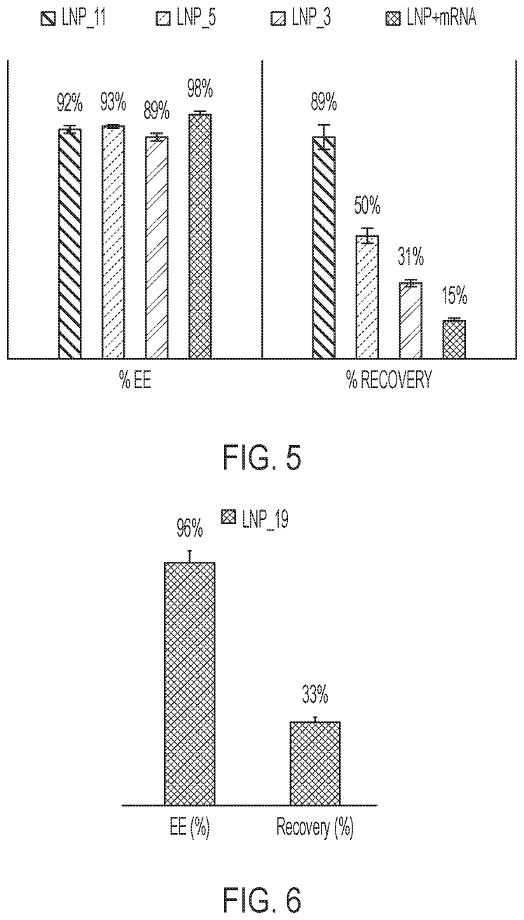

Due to the aspect ratio of polynucleotides, the polynucleotides behave in ways that are unexpected based on conventional filtration techniques that are based on the molecular weight of components of a feed. For example and as depicted conceptually in FIG. 3, the long polynucleotides 202 can pass through a filter 306 having pores 308 that are smaller than specified for molecules having the long polynucleotide's molecular weight. Without being bound by any particular theory, Applicant believes that this is attributable to the narrow cross-sectional dimension of the long polynucleotides 202 relative to the larger cross-sectional dimension of LNPs 204 encapsulating the long polynucleotides 202.

The invention includes a method of separating polynucleotides from a composition (e.g., purifying mRNA from a LNP). In some embodiments, separating polynucleotides comprises separating short polynucleotides (e.g., less than 100 bps in length) as well as separating long polynucleotides (e.g., greater than 1,000 bps in length). In one embodiment, the long polynucleotide has a length of 1 kb (kilobases) or greater. Although filtration of long polynucleotides is unexpected, embodiments of the invention capable of separating long polynucleotides would also be able to separate short polynucleotides.

### TFF

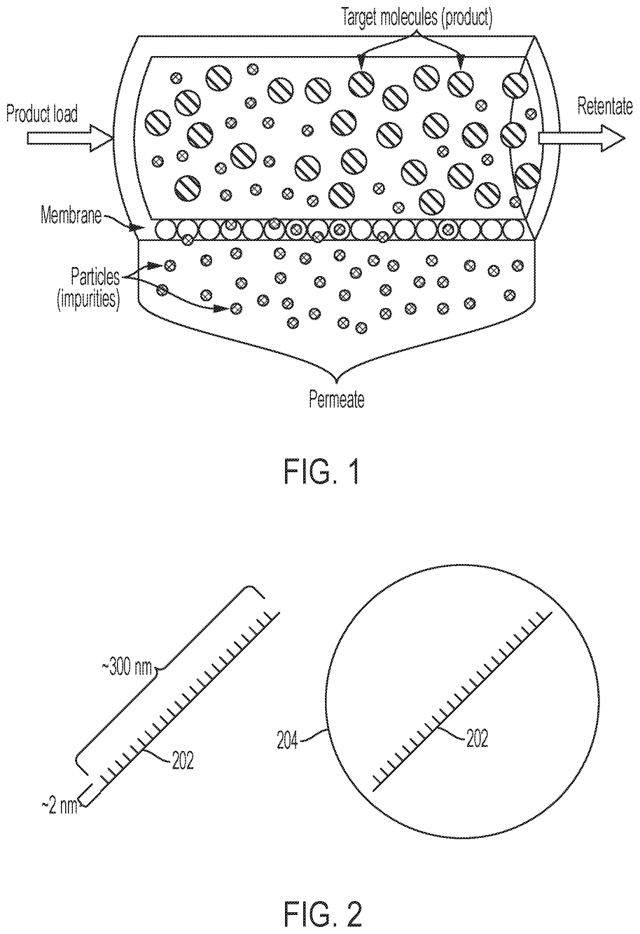

A variety of filtering architectures can be utilized. In some embodiments (colloquially known as “dead-end filtration”), the pore-containing filter is at an end of a container and fluid is pressed against the filter (e.g., by gravity or other pressure source). In other embodiments, the filter is a tangential flow filtration device in which the filter is positioned laterally to the feed flow direction. Such a device is depicted in FIG. 1. Tangential flow filtration (TFF), also referred to as cross-flow filtration, is a type of filtration wherein the material to be filtered is passed tangentially across a filter rather than through it. In TFF, undesired permeate passes through the filter, while the desired retentate passes along the filter and is collected downstream. It is important to note that the desired material is typically contained in the retentate in TFF, which is the opposite of what one normally encounters in traditional-dead end filtration.

Tangential- or cross-flow filters can have a variety of architectures including hollow fiber, spiral wound, and flat plate. Suitable filters are available from a variety of sources including; Cole-Parmer of Vernon Hills, Ill., Millipore Corporation of Billerica, Mass., and Repligen Corporation of Waltham, Mass. Depending upon the material to be filtered, TFF is usually used for either microfiltration or ultrafiltration Microfiltration is typically defined as instances where the filter has a pore size of between 0.05 μm and 1.0 μm, inclusive, while ultrafiltration typically involves filters with a pore size of less than 0.05 μm. Pore size also determines the nominal molecular weight limits (NMWL), also referred to as the molecular weight cut off (MWCO) for a particular filter, with microfiltration membranes typically having NMWLs of greater than 1,000 kilodaltons (kDa) and ultrafiltration filters having NMWLs of between about 1 kDa and about 1,000 kDa.

Tangential-flow filtration can be implemented on industrial scale and can include system(s) adapted, configured, and/or programmed to periodically take steps to delay or prevent fouling of the filter. For example, the system can backwash the filter by periodically inverting the transmembrane pressure (TMP), providing alternating tangential flow, cleaning-in-place with detergents, reactive agents, and alkalis, and periodically closing or reducing flow from the permeate outlet.

A variety of filtration systems, methods, and parameters are described in Herb Lutz, Ultrafiltration for Bioprocessing. Development and Implementation of Robust Processing (2015).

One or more process variables can be important in the TFF process, transmembrane pressure, feed rate, flow rate or flux of the permeate. The transmembrane pressure is the force that drives fluid through the filter, carrying with it permeable molecules. In some embodiments, the transmembrane pressure is between about 0.5 and about 10 pounds per square inch (psi), inclusive.

Elevated pressure can be measured across the filter. Thus, the elevated pressure can be a pressure gradient generated in-whole or in-part by a vacuum on the opposite side from the composition or a back-pressure valve on the retentate. For example, elevated pressure can be generated by fluid flow velocity, syringes, pumps (e.g, peristaltic pumps), and the like.

The elevated pressure can be a pressure gradient between about 0.5 and about 10 pounds per square inch (psi) (between about 3,500 and about 70,000 Pascal).

Shear rate is the rate at which a progressive shearing deformation is applied to some material. Exemplary shear rates are between about 1,000 and about 20,000 s⁻¹ (e.g., about 7,000 s⁻¹.

The feed rate (also known as the crossflow velocity) is the rate of the solution flow through the feed channel and across the filter. The feed rate determines the force that sweeps away molecules that may otherwise clog or foul the filter and thereby restrict filtrate flow. In some embodiments, the feed rate is between about 50 and about 500 mL/minute. In some embodiments, the feed rate is between about 50 and about 400 mL/minute. In some embodiments, the feed rate is between about 50 and about 300 mL/minute. In some embodiments, the feed rate is between about 50 and about 200 mL/minute. In some embodiments, the feed rate is between about 75 and about 200 mL/minute. In some embodiments, the feed rate is between about 100 and about 200 mL/minute. In some embodiments, the feed rate is between about 125 and about 175 mL/minute. In some embodiments, the feed rate is about 130 mL/minute. In some embodiments, the feed rate is between about 60 mL/min and about 220 mL/min. In some embodiments, the feed rate is about 60 mL/min or greater. In some embodiments, the feed rate is about 100 mL/min or greater. In some embodiments, the feed rate is about 150 mL/min or greater. In some embodiments, the feed rate is about 200 mL/min or greater. In some embodiments, the feed rate is about 220 mL/min or greater.

In some embodiments, the tangential flow filtration is performed at a feed rate of approximately 100-200 mL/minute (e.g., approximately 100-180 mL/minute, 100-160 mL/minute, 100-140 mL/minute, 110-190 mL/minute, 110-170 mL/minute, or 110-150 mL/minute) and/or a flow rate of approximately 10-50 mL/minute (e.g., approximately 10-40 mL/minute, 10-30 mL/minute, 20-50 mL/minute, or 20-40 mL/minute). In some embodiments, the tangential flow filtration is performed at a feed rate of approximately 100, 110, 120, 130, 140, 150, 160, 170, 180, 190, or 200 mL/minute and/or a flow rate of approximately 10, 20, 30, 40, or 50 mL/minute.

The flow rate of the permeate is the rate at which the permeate is removed from the system. For a constant feed rate, increasing permeate flow rates can increase the pressure across the filter, leading to enhanced filtration rates while also potentially increasing the risk of filter clogging or fouling. The principles, theory, and devices used for TFF are described in Michaels et al, “Tangential Flow Filtration” in Separations Technology, Pharmaceutical and Biotechnology Applications (W. P. Olson, ed., Interpharm Press, Inc., Buffalo Grove, 111, 1995). See also U.S. Pat. Nos. 5,256,294 and 5,490,937 for a description of high-performance tangential flow filtration (HP-TFF), which represents an improvement to TFF.

In some embodiments, the flow rate is between 10 and 100 mL/minute. In some embodiments, the flow rate is between about 10 and about 90 mL/minute. In some embodiments, the flow rate is between about 10 and about 80 mL/minute. In some embodiments, the flow rate is between about 10 and about 70 mL/minute. In some embodiments, the flow rate is between about 10 and about 60 mL/minute. In some embodiments, the flow rate is between about 10 and about 50 mL/minute. In some embodiments, the flow rate is between about 10 and about 40 mL/minute. In some embodiments, the flow rate is between about 20 and about 40 mL/minute. In some embodiments, the flow rate is about 30 mL/minute.

In some embodiments, flow rates to accommodate large (commercial) scale purification entail the tangential flow filtration is performed at a feed rate of approximately 10-200 L/minute. (e.g., approximately 10-180 L/minute, 100-160 L/minute, 100-140 L/minute, 110-190 L/minute, 110-170 L/minute, or 110-150 L/minute) and/or a flow rate of approximately 10-50 L/minute (e.g., approximately 10-40 L/minute, 10-30 L/minute, 20-50 L/minute, or 20-40 L/minute). In some embodiments, the tangential flow filtration is performed at a feed rate of approximately 100, 110, 120, 130, 140, 150, 160, 170, 180, 190, or 200 L/minute and/or a flow rate of approximately 10, 20, 30, 40, or 50 L/minute.

Flux is a measure of the magnitude of flow across the membrane. Exemplary flux values are between about 20 and about

$$200 \frac{L}{h \cdot {m}^{2}}$$

(e.g., between about 25 and about

$$55 \frac{L}{h \cdot {m}^{2}} ) .$$

### Pores

A filter can include a plurality of pores. Pores can have a regular, uniform shape such as a cylindrical or a rectangular profile, and the like, or can have an irregular profile. The pores can have a nanoscale and may not be capable of dimensional measurement. Instead, the pores may be measured based on filtration performance, e.g., based on what molecular weight particles will and/or will not pass through the filter.

The term “molecular weight cut-off” or MWCO refers to the lowest molecular weight solute (in daltons) in which x % (typically 90) of the solute is retained by a membrane. As described herein, filters used in the invention described herein may have any of a variety of pore sizes. Pore size determines the nominal molecular weight limits (NMWL), also referred to as the molecular weight cut off (MWCO) for a particular filter, with microfiltration membranes typically having NMWLs of greater than about 1,000 kilodaltons (kDa) and ultrafiltration filters having NMWLs of between about 1 kDa and about 1,000 kDa.

In some embodiments, a filter will have a NMWL of between about 100 kDa and about 1,500 kDa. In some embodiments, a filter will have a NMWL of between about 500 kDa and about 1,000 kDa. In some embodiments, a filter will have a NMWL between about 600 kDa and about 800 kDa. In some embodiments, a filter has a NMWL of about 750 kDa.

Without being bound by theory, Applicant believes that filters having an MWCO of about 1,000 kDa and/or a maximum cross-sectional dimension between about 100 nm and about 150 nm are particularly advantageous for filtering long polynucleotides. This is significantly lower than the 1,500 kDa molecular weight of 4,500 kbp mRNA. In certain embodiments, the maximum cross-sectional dimension of the filter is about, 99, 100, 110, 120, 130, 140, or 150 nm, and any and all values in between or has a MWCO of about 700, 750, 800, 850, 900, 950, 1000, 1050, and any and all values in betwee.

### EXAMPLES

### Example 1: Synthesis of mRNA

This Example demonstrates mRNA synthesis.

mRNA is typically thought of as the type of RNA that carries information from DNA to the ribosome. The existence of mRNA is typically very brief and includes processing and translation, followed by degradation. In some embodiments, large scale quantities of mRNA may need to be purified away from IVT (in vitro synthesis) components. In some embodiments, purified mRNA may further be utilized in subsequent preparations, e.g., polynucleotide loaded nanoparticles. In the following Example, mRNA is synthesized.

Briefly. IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. The presence of these reagents is undesirable in the final product and may thus be referred to as impurities.

In the following example, mRNA is synthesized via in-vitro transcription from a linearized DNA template. To produce the desired mRNA precursor (IVT) construct, a mixture of 100 μg of linearized DNA, rNTPs (3.33 mM), DTT (10 mM), T7 RNA polymerase, RNAse Inhibitor, Pyrophosphatase and reaction buffer (10×, 800 mM Hepes (pH8.0), 20 mM Spermidine, 250 mM MgCl2, pH 7.7) is prepared with RNase-free water to a final volume of 2.24 mL. The reaction mixture is incubated at 37° C. for a range of time between 20 minutes and 120 minutes. Upon completion, the mixture is treated with DNase I for an additional 15 minutes and quenched accordingl.

The IVT mRNA is diluted in 4× volume of PBS pH 7.4 immediately after quenching.

### Example 2: Preparation of Protein/Polynucleotide Mixtures

This Example demonstrates preparation of a protein and polynucleotide mixture.

RNA and protein are present in whole cell lysates, nuclear, and/or cytoplasmic fractions. Isolation of large scale, high quality RNA is becoming more popular. In some circumstances, complementary proteins from the same preparations are also required Usually these preparations are performed using different separation protocols, wasting large quantities of the preparation to purify one component. The addition of independent preparations and separation protocols is costly and time-consuming. The following Example demonstrates preparation of a protein and polynucleotide mixture for separation.

Tissue or cultured cells are homogenized in ice-cold Cell Disruption Buffer (2M KCl, 1M MgCl₂, 1M pH 7.5 Tris-Cl, RNase-free H₂O) to prepare a total cell lysate. Homogenization is performed quickly on ice and in the presence of detergent (1M DTT). The lysate is diluted in 4× volume of PBS pH 7.4 immediately after homogenization.

### Example 3: Preparation of Polynucleotide Loaded Nanoparticles

This Example demonstrates preparation of lipid-based nanoparticles (LNPs).

Lipid-based nanoparticles (LNPs) are an effective delivery vehicle for in-vivo delivery of nucleic acid-based therapeutics, including RNA-based ones. Sterically stabilizing lipids may include PEG-lipid conjugates (i.e. PEG-DMG), which cover the surface of the LNPs and shield overall surface charges (positive or negative), make the surface hydrophilic, and in in vivo applications, prevent opsonization to increase longevity in the blood.

Effective encapsulation of nucleic acids is important since free (or naked) RNA molecules have significantly shorter half-time in the blood circulation-usually measurable in minutes. Thus, removal of free/non-encapsulated nucleic acids and purification processes of LNPs are important for effective drug delivery of mRNAs (e.g., those having >1000 bases).

The LNP formulations were prepared by mixing a lipid ethanol solution (organic phase) with mRNA (large mRNA with 4500 kb, molecular weight is approx. 1500 kDa) having a pH 4.5 in an aqueous buffer (aqueous phase) at predetermined aqueous-to-organic phase volume ratios of 2:1 or 3:1 and flow rates varying from 2 ml/min to 8 ml/min in a microfluidics-based preparation chip. (Ratios between about 1:1 and about 4.1 and/or flow rates between about 1 ml/min to about 12 ml/min are also possible.) The resulting LNP dispersion included ethanol, whose percentage was dependent on the mixing ratio.

The LNP dispersion was diluted in 10× volume of PBS pH 7.4 immediately upon collection.

### Example 4: Pressure Filtration of Polynucleotide Mixtures

This Example demonstrates separation of polynucleotides from other components in a mixture.

Purification methods are typically based on the removal of polynucleotides through filtration with membranes having MWCO sizes of at least 1500 kDa for 4500 kb length polynucleotides. High MWCO membranes, such as 1000 kDa, have pore sizes as big as 100 nm, thus other components leak out from the membrane, causing material loss and impure separations.

Currently, two main buffer exchange/purification/concentration methods are used in the technique of processing mixtures with polynucleotides. The first involves the dialysis of the mixtures against PBS for buffer exchange and removal of ethanol from the dispersion. However, when large polynucleotides are present (˜1500 kDa), this method cannot be used since the largest MWCO for dialysis membranes is 300 kDa, far smaller than the polynucleotides. While high pressures applied in closed-chamber systems might push large polynucleotides through the 300 kDa membrane, the pressure levels required for effective purification will negatively impact stability of additional preparations in the mixture that are the main products to be purified, such as but not limited to proteins and/or LNPs, and cause potential breakdown when passing through the membrane. Furthermore, the removal process takes significantly longer time, generally requiring 24-48 h of dialysis.

The second method involves using centrifugal ultrafiltration devices. In this method, the mixture components are pushed through a membrane by applying extreme g-force. But if the g-force is too low, the removal of free polynucleotides is limited. If the force is high, then stability of the components is negatively affected, i.e., the components are crushed. Moreover, the solution that contains the mRNA and products to be purified will be stationary at the membrane contact surface, which can cause clogging of the pores in such membranes. The following Example demonstrates a purification method with a membrane having a MWCO size selected to allow the non-encapsulated polynucleotides to be separated while keeping the other components inside the membrane. The ethanol removal and buffer exchange are performed on a tangential flow filtration (TFF) device with a molecular weight cut off (MWCO) of 750 kDa. Moreover, the method allows the buffer exchange/purification/concentration steps to be combined in one method and device.

The final products of Example 1 or Example 2 may be used as a substitute to the LNP dispersion of Example 3 in the following.

The LNPs dispersion of Example 3 was concentrated to 1 ml volume (initial volume depends on the batch size, usually between 12-100 ml after mixing in PBS). Then, the separation of polynucleotides from LNPs was performed by washing the mixtures three times with PBS, each time with 10 ml volume. Each time, the mixtures were concentrated to 1 ml volume.

The buffer exchange/purification/concentration was achieved by passing the mixture through the TFF device by applying pressure, either manually via syringes or automatically via a system equipped with pumps (e.g., peristaltic pumps) and pressure sensors, in the distal ends of the device. During each pass in the membrane, a portion of the dispersion phase (aqueous phase) leaked out through the hollow fibers, carrying the non-encapsulated mRNA molecules with it (FIG. 1). The transmembrane pressure exerted did not produce damage to the membrane, kept the LNP structure intact, and was in the range of about 0 to 30 psi (about 0 to 205,000 Pascal). The final volume of concentrated LNPs was 0.5 ml.

The TFF membrane selected had a MWCO size of 750 kDa-half the size that would be predicted for 4500 kb mRNA and free mRNA molecules of this size to stay inside the membrane. However, the selected pressure range pushed the mRNAs through pores half the size expected for their molecular weight. Moreover, the relationship between the applied pressure and the MWCO size can be applied to all the previous preparations for TTF-based polynucleotide purification methods.

### Example 5: Characterization of LNPs

This Example demonstrates characterization of lipid-based nanoparticles (LNPs).

LNPs encapsulating mRNA were prepared as described in Example 3. They were subjected to the purification process as described in Example 4 prior to characterization.

FIG. 4 shows the washing/removal efficacy of the 1500 kDa mRNA as a function of washing volume/cycle number. As show in the graph, the first pass from the TFF membrane reduced the mRNA concentration in the retentate 5-times. The PBS volume to wash the mRNA solution was 5 ml. After 3 washes (15 ml), the remaining mRNA in the retentate dropped to negligible amounts and 4 wash cycles (20 ml total) completely removed the mRNA. Table 1 also summarizes the mRNA concentrations in the retentate.

TABLE 1
Average mRNA concentrations (ng/μl) in retentate

| Sample IDs | Avg. (ng/μl) |
|---|---|
| 1. mRNA sample: 45 μg/ml | 43.24 |
| 2. mRNA load 1 (~9 μg/ml) | 7.725 |
| 3. Wash 1 | 3.54 |
| 4. Wash 2 | 0.78 |
| 5. Wash 3 | 0.205 |
| 6. Wash 4 | 0 |
| 7. Wash 5 | −0.82 |
| 8. Load Recovery | −0.355 |

Following validation of the protocol, the LNPs were buffered/purified/concentrated and the EE and recovery were determined. The encapsulation efficiency and the total recovery of the mRNA was determined by a fluorescent RNA quantification assay (Life Technologies) according to the manufacturer s instructions. Briefly, aliquots of LNP solution were diluted 1:1 in TE buffer (measuring the non-encapsulated free mRNA) or 1:1 in TE buffer containing 2% Triton X-100 (measuring total mRNA, both encapsulated and non-encapsulated). Assay reagent was added to each sample and fluorescent signal was quantified. mRNA concentration was calculated using a calibration curve prepared with known concentrations of mRNA. The assay measured the non-encapsulated or LNP surface bound mRNAs in the TE-diluted samples. TE-Triton X-100 (a strong detergent) broke the LNPs apart and released the encapsulated mRNA from the LNPs, thus the measured mRNA concentration was the total mRNA in the LNP sample. FIGS. 3 and 4 show that the method purified the LNPs encapsulated sgRNA or mRNA regardless of high or low recovery values (FIG. 3). Changing the formulation parameters, such as lipid compositions, flow rates, and mixing ratio of aqueous:organic phases allowed higher recovery rates with higher EE values, indicating removal of free non-encapsulated mRNA (FIG. 4).

Particle sizes of the LNPs were measured using dynamic light scattering (DLS). Briefly, the formulations were diluted in an isotonic buffer with the same ionic equivalence and pH value as the LNP dispersion at ratios varying from 1.20 to 1.1000 v/v. The sample was injected into the sample cell of the instrument. Following temperature equilibration for 120 seconds, backscattered light from the 488 nm laser was collected. The average particle size and polydispersity values of minimum three acquisitions were reported for each LNP formulation.

Regardless of the formulation and preparation parameters, LNP purification to remove non-encapsulated mRNA impacted encapsulation efficiency and recovery.

The following data show that at low TMP and at high TMP, there is less efficient removal of unencapsulated mRNA relative to an intermediate TMP. This is likely due to achieving a balance between the pressure required to force mRNAs through the pores and preventing membrane fouling at higher pressures.

TABLE 2
Purification of unencapsulated mRNA from an LNP suspension
by tangential flow filtration as a function of TMP

| TMP (PSI (Pascal)) | % Unencapsulated RNA |
|---|---|
| 1.9 (13,000) | 11% |
| 3.2 (22,000) | <5% |
| 7.8 (54,000) | 9% |

### Example 6: Analysis of Purified Polynucleotide

This Example demonstrates analysis of the polynucleotides separated from the other components.

In some embodiments, purified polynucleotides are collected and the purified polynucleotides are analyzed for impurities. In the following Example, purified polynucleotides in the preparations of Example 1 or Example 2 and pressure filtered as described in Example 4, may be analyzed as in the following Example.

Coomassie-stained protein gels may be performed to determine the presence of residual proteins present before and after purifications. In some instances. BCA assays may be performed as well.

mRNA size and integrity may be assessed via gel electrophoresis. Either 1.0% agarose gel or Invitrogen E-Gel precast 1.2% agarose gels may be employed. mRNA is loaded at 1.0-1.5 μg quantities per well. Upon completion, messenger RNA bands are visualized using ethidium bromide.

In vitro transfections of control luciferase mRNA may be performed using HEK293T cells. Transfections of 1 μg of each mRNA construct are performed in separate wells using a lipid based-transfection reagent according to manufacturer's protocol. Cells are harvested at selected time points (e.g., 4 hour, 8 hour, etc.) and respective protein production is analyzed. For FFL mRNA, cell lysates are analyzed for luciferase production via bio luminescence assays.

In Examples measuring a control fluorescent RNA, a bioluminescence assay may be conducted using a Promega Luciferase Assay System (Item #El 500) The Luciferase Assay Reagent is prepared by adding Luciferase Assay Buffer to Luciferase Assay Substrate and mixing via a vortex.

Purified polynucleotides from Example 4 are loaded onto a 96-well plate followed by plate control to each sample. Separately, Luciferase Assay Reagent (prepared as described above) is added to each well of a 96-well flat bottomed plate. Each plate is then inserted into the appropriate chambers using a Molecular Device Flex Station instrument and the luminescence (measured in relative light units (RLU)) is measured.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of separating long polynucleotides from a composition, the method comprising:
introducing a composition including one or more long polynucleotides into a container including at least one boundary defined by a filter comprising a plurality of pores, wherein the pores have a smaller cross-sectional dimension than specified for the long polynucleotide's molecular weight; and
applying elevated hydraulic pressure to the composition;
thereby causing at least some of the one or more long polynucleotides to pass through the pores.

2. A method of separating a long polynucleotide from a composition, the method comprising:
(a) providing a composition comprising a long polynucleotide;
(b) disposing the composition within a container including at least one boundary comprising a plurality of pores having a smaller cross-sectional dimension than specified for a molecular weight of the long polynucleotide; and
(c) subjecting the composition to elevated hydraulic pressure within the container under conditions to cause the long polynucleotide to pass through the pores;
thereby separating the long polynucleotide from the composition.

3. The method of claim 1 or 2, wherein the long polynucleotides have a length of about 1 kilobase or greater.

4. The method of claim 1 or 2, wherein the long polynucleotides have a length selected from the group consisting of: ≥950 kilobase, ≥900 kilobase, ≥850 kilobase, ≥800 kilobase, ≥750 kilobase, ≥700 kilobase, ≥650 kilobase, and ≥600 kilobase.

5. The method of claim 1 or 2, wherein the composition further comprises a second component.

6. The method of claim 4, wherein the second component does not pass through the pores.

7. The method of claim 1 or 2, wherein:
the filter is positioned within the container between an inlet and an outlet; and
the composition is flowed over the filter.

8. The method of claim 7, wherein the composition is flowed laterally over the filter.

9. The method of claim 1 or 2, wherein the elevated hydraulic pressure is between about 0.1 and about 30 pounds per square inch (psi), inclusive.

10. The method of claim 1 or 2, wherein the elevated hydraulic pressure is below a pressure that would impact the stability of other components of the composition

11. The method of claim 1 or 2, wherein the long polynucleotides are sheared during passage through the pores.

12. The method of claim 1 or 2, wherein the pores have an effective molecular weight cutoff of about 1,000 kDa.

13. The method of claim 1 or 2, wherein the pores have a maximum cross-sectional dimension between about 50 nm and about 150 nm.
